(19) United States
(12) Patent Application Publication
(10) Pub. No.: US 2014/0200488 A1
(43) Pub. Date: Jul. 17, 2014

(54) METHOD OF COOLING ULTRASOUND TREATMENT APPARATUS AND ULTRASOUND TREATMENT APPARATUS USING THE SAME

(71) Applicant: SAMSUNG ELECTRONICS CO., LTD., Suwon-si (KR)

(72) Inventors: Joon-ho SEO, Seoul (KR); Won-chul BANG, Seongnam-si (KR); Do-kyoon KIM, Seongnam-si (KR); Sang-hyun KIM, Hwaseong-si (KR)

(73) Assignee: SAMSUNG ELECTRONICS CO., LTD., Suwon-si (KR)

(21) Appl. No.: 14/072,416

(22) Filed: Nov. 5, 2013

(30) Foreign Application Priority Data
Jan. 15, 2013 (KR) 10-2013-0004467

(51) Int. Cl. A61N 7/00

(52) U.S. Cl. CPC A61N 7/00
USPC 601/3

(57) ABSTRACT

An ultrasound treatment apparatus includes a transducer configured to irradiate ultrasound waves onto an object, and a membrane disposed in an irradiation direction of the ultrasound waves and to form, with the transducer, a space in which a cooling fluid circulates. The apparatus further includes a first temperature sensor configured to measure a temperature of a skin of a patient contacting the membrane, and a second temperature sensor configured to measure a temperature of the transducer. The apparatus further includes a cooling unit configured to circulate the cooling fluid based on the measured temperatures.

### CROSS-REFERENCE TO RELATED APPLICATION

This application claims the benefit under 35 USC 119(a) of Korean Patent Application No. 10-2013-0004467, filed on Jan. 15, 2013, in the Korean Intellectual Property Office, the entire disclosure of which is incorporated herein by reference for all purposes.

### BACKGROUND

1. Field

The present disclosure relates methods of cooling an ultrasound treatment apparatus and the ultrasound treatment apparatus using the same.

2. Description of the Related Art

With the development of medical science, a local treatment for tumors has used invasive surgery, in addition to non-invasive surgery. A high-intensity focused ultrasound (HIFU) treatment of non-invasive surgery uses sound waves and thus is harmless to humans. Therefore, the HIFU treatment has been widely used. The HIFU treatment refers to a treatment method of irradiating and focusing high-intensity ultrasound waves on a lesion of a human body to necrose a lesion tissue. In other words, if a lesion occurs in a tissue of a subject, an ultrasound treatment apparatus irradiates treatment ultrasound waves onto the lesion to form a burn, and a diagnostic ultrasound apparatus acquires ultrasound images from the tissue with the lesion to diagnose whether a treatment is completed.

In the HIFU treatment, a transducer, which irradiates high-intensity, consecutive ultrasound waves to generate ultrasound waves, is heated. Therefore, studies have been conducted on preventing a rise in a temperature of the transducer.

### SUMMARY

In one general aspect, an ultrasound treatment apparatus includes a transducer configured to irradiate ultrasound waves onto an object, and a membrane disposed in an irradiation direction of the ultrasound waves and to form, with the transducer, a space in which a cooling fluid circulates. The apparatus further includes a first temperature sensor configured to measure a temperature of a skin of a patient contacting the membrane, and a second temperature sensor configured to measure a temperature of the transducer. The apparatus further includes a cooling unit configured to circulate the cooling fluid based on the measured temperatures.

The first temperature sensor may be disposed on a side of the membrane directly contacting the skin, and inside an ultrasound beam formed by an irradiation path of the ultrasound waves.

The first temperature sensor may include first temperature sensors disposed at predetermined intervals on the membrane, and the second temperature sensor may include second temperature sensors disposed at predetermined intervals on the transducer.

A size of the first temperature sensor may be less than or equal to ¼ of a size of a wavelength of the ultrasound waves.

The cooling unit may include a cooler controller configured to compare the measured temperature of the transducer or the measured temperature of the skin with a predetermined temperature that is a cooling criterion, and determine a temperature and/or a flow rate of the cooling fluid based on a result of the comparison, and a cooler configured to circulate the cooling fluid based on the determined temperature and/or the determined flow rate.

The cooler controller may be configured to compare a higher temperature among the temperature of the skin and the temperature of the transducer with the predetermined temperature, decrease the temperature of the cooling fluid, and increase the flow rate, in response to the higher temperature being greater than the predetermined temperature, decrease the flow rate in response to the higher temperature being less than the predetermined temperature, and maintain a current circulation of the cooling fluid in response to the higher temperature being equal to the predetermined temperature.

The predetermined temperature may be a lower temperature among a lowest temperature causing pain due to feeling heat and a limit temperature for a normal operation of the transducer.

The cooler controller may be configured to compare the temperature of the skin and the temperature of the transducer with first and second predetermined temperatures, respectively, decrease the temperature of the cooling fluid, and increase the flow rate, in response to each of the temperature of the skin and the temperature of the transducer being greater than the first or second predetermined temperature, maintain a current circulation of the cooling fluid in response to each of the temperature of the skin and the temperature of the transducer being equal to the first or second predetermined temperature, and decrease the flow rate in other cases.

The first predetermined temperature may be a lowest temperature causing pain due to feeling heat, and the second predetermined temperature may be a limit temperature for a normal operation of the transducer.

The cooling unit may include a first data collector configured to acquire one or more temperatures of the skin that are measured by the first temperature sensor, and obtain, as a representative value of the temperature of the skin, an average of the acquired one or more temperatures of the skin, or a highest temperature among the acquired one or more temperatures of the skin, or a temperature value of the skin that is measured by a temperature sensor at a position selected by a user. The cooling unit may include a second data collector configured to acquire one or more temperatures of the transducer that are measured by the second temperature sensor, and obtain, as a representative value of the temperature of the transducer, an average of the acquired one or more temperatures of the transducer, or a highest temperature among the acquired one or more temperatures of the transducer, or a temperature value of the transducer that is measured by another temperature sensor at another position selected by the user.

In another general aspect, there is provided a method of cooling an ultrasound treatment apparatus including a transducer irradiating ultrasound waves onto an object, and a membrane forming, with the transducer, a space in which a cooling fluid circulates. The method includes measuring a temperature of a skin of a patient contacting the membrane, measuring a temperature of the transducer, and circulating the cooling fluid based on the measured temperatures.

The measuring of the temperature of the skin may include measuring the temperature of the skin at a side of the membrane directly contacting the skin, and inside an ultrasound beam formed by an irradiation path of the ultrasound waves.

The measuring of the temperature of the skin may include measuring temperatures of the skin, and the measuring of the temperature of the transducer may include measuring temperatures of the transducer. The method may further include obtaining, as a representative value of the temperature of the skin, an average of the measured temperatures of the skin, or a highest temperature among the measured temperatures of the skin, or a temperature value of the skin that is measured by a temperature sensor at a position selected by a user, and obtaining, as a representative value of the temperature of the transducer, an average of the measured temperatures of the transducer, or a highest temperature among the measured temperatures of the transducer, or a temperature value of the transducer that is measured by another temperature sensor at another position selected by the user.

A non-transitory computer-readable storage medium may store a program including instructions to cause a computer to perform the method.

In still another general aspect, an apparatus includes a transducer configured to irradiate an ultrasound beam onto a subject, and a membrane configured to form, with the transducer, a space in which a cooling fluid circulates, and contact the subject.

Other features and aspects will be apparent from the following detailed description, the drawings, and the claims.

### BRIEF DESCRIPTION OF THE DRAWINGS

FIG. 1 is a block diagram illustrating an example of an ultrasound treatment and diagnosis system.

FIG. 2 is a block diagram illustrating an example of an ultrasound treatment apparatus.

FIG. 3 is a perspective view illustrating an example of an external appearance of a header of the ultrasound treatment apparatus.

FIGS. 4A and 4B are cross-sectional views illustrating examples of the header of the ultrasound treatment apparatus.

FIG. 5 is a view illustrating an example of a heat conduction generated in the header of the ultrasound treatment apparatus.

FIG. 6 is a flowchart illustrating an example of a method of cooling an ultrasound treatment apparatus.

FIG. 7 is a detailed flowchart illustrating an example of operation 620 of FIG. 6.

FIG. 8 is a detailed flowchart illustrating an example of operation 630 of FIG. 6.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. Also, descriptions of functions and constructions that are well known to one of ordinary skill in the art may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

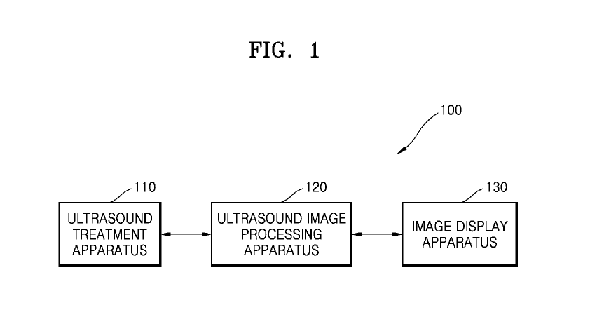

FIG. 1 is a block diagram illustrating an example of an ultrasound treatment and diagnosis system 100. Referring to FIG. 1, the ultrasound treatment and diagnosis system 100 includes an ultrasound treatment apparatus 110, an ultrasound image processing apparatus 120, and an image display apparatus 130. Although FIG. 1 schematically illustrates an entire structure of the ultrasound treatment and diagnosis system 100, the ultrasound treatment and diagnosis system 100 may further include other general-purpose elements besides the elements of FIG. 1. Elements constituting the ultrasound treatment and diagnosis system 100 may not be physically separated from one another but may be integrated with one another differently from shown in FIG. 1.

If a lesion such as a tumor occurs in a subject, the ultrasound treatment and diagnosis system 100 irradiates treatment ultrasound waves onto the lesion through the ultrasound treatment apparatus 110 to form a burn. The ultrasound treatment and diagnosis system 100 irradiates diagnostic ultrasound waves onto a tissue with the lesion, and receives an echo signal to generate and display ultrasound images of the tissue with the lesion, thereby helping experts such as doctors determine whether a treatment for the lesion is completed.

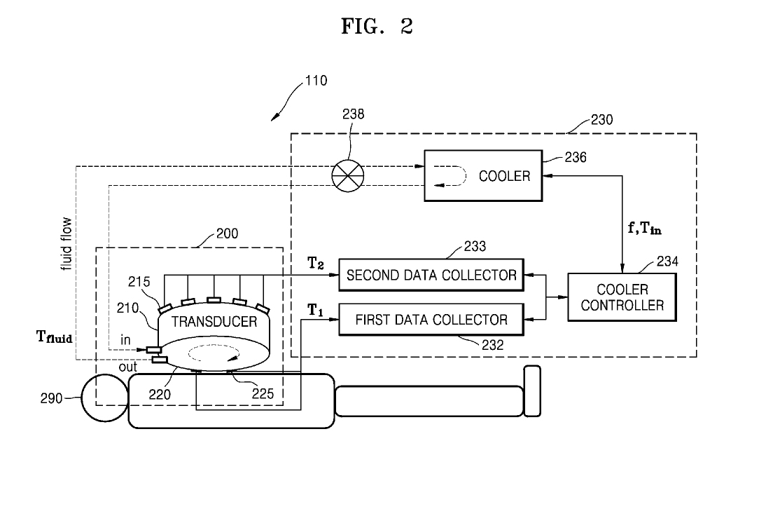

The ultrasound treatment apparatus 110 irradiates the treatment ultrasound waves onto the lesion to necrose the lesion in order to treat the subject. High-intensity focused ultrasound (HIFU) waves may be used as the treatment ultrasound waves. A diagnostic probe to irradiate the diagnostic ultrasound waves may be integrated with the ultrasound treatment apparatus 110 or may be constituted as an additional ultrasound diagnostic apparatus. The diagnostic probe irradiates the diagnostic ultrasound waves onto the tissue with the lesion, and receives the echo signal of the irradiated diagnostic ultrasound waves. The echo signal is used to generate the ultrasound images and monitor changes of a temperature of the tissue with the lesion. An operation and a function of an ultrasound treatment apparatus is described below in detail with reference to FIG. 2.

The ultrasound image processing apparatus 120 receives a command from a user to generate a signal to control the ultrasound treatment apparatus 110. The ultrasound image processing apparatus 120 generates the ultrasound images of the tissue with the lesion based on the echo signal received by the diagnostic probe.

The image display apparatus 130 receives signals of the generated ultrasound images from the ultrasound image processing apparatus 120. The image display apparatus 130 displays an ultrasound image or a temperature image on a display based on the received signals.

FIG. 2 is a block diagram illustrating an example of the ultrasound treatment apparatus 110. Referring to FIG. 2, the ultrasound treatment apparatus 110 includes a header 200 and a cooling unit 230. The header 200 includes a transducer 210, a membrane 220, one or more first temperature sensors 225, and one or more second temperature sensors 215. The cooling unit 230 includes a first data collector 232, a second data collector 233, a cooler controller 234, a cooler 236, and a pump 238. The ultrasound treatment apparatus 110 may further include other general-purpose elements besides the elements of FIG. 2.

The transducer 210 generates ultrasound waves, and irradiates the ultrasound waves onto an object. The object refers to a tissue including a lesion in a body of a patient 290. The transducer 210 includes a piezoresonator to convert electric energy into ultrasound waves, or convert ultrasound waves into electric energy. The transducer 210 may include a plurality of piezoresonators, and thus may include an array form in which dozens of piezoresonators are connected, an n×m matrix form, or a form in which piezoresonators are close to one another in a circle. If the transducer 210 irradiates HIFU waves, the transducer 210 may include an arch shape or a concave plate shape such as a parabolic antenna to focus focal points of the irradiated HIFU waves on a location. Several separated transducers 210 may irradiate ultrasound waves onto a focal point. The transducer 210 is installed in a housing (not shown). Ultrasound beams including con-beam shapes may be formed by a path through which the irradiated ultrasound waves pass.

The membrane 220 is positioned in a direction in which the transducer 210 irradiates the ultrasound waves, and forms a space in which a cooling fluid circulates, along with the transducer 210. The membrane 220 is a membrane into which the cooling fluid does not penetrate, and may be formed of an elastic material. If the membrane 220 is formed of an elastic material, the membrane 220 may increase a contact ratio of the membrane 220 contacting the skin of the patient 290. A side of the membrane 220 contacts the skin of the patient 290, and another side of the membrane 220 contacts the cooling fluid.

Each of the first temperature sensors 225 measures a temperature of the skin of the patient 290 contacting the membrane 220. If the transducer 210 irradiates the ultrasound waves onto lesion, the ultrasound waves penetrate the skin of the patient 290. Therefore, the skin of the patient 290 contacting the ultrasound treatment apparatus 110 is heated by consecutive irradiations of the ultrasound waves, and thus the temperature of the skin increases. Each of the first temperature sensors 225 monitors the temperature of the skin so that the patient 290 is not damaged by heat.

Each of the first temperature sensors 225 is positioned on the side of the membrane 220 directly contacting the skin of the patient 290 and inside the ultrasound beams formed by the path through which the ultrasound waves irradiated from the transducer 210 pass. The one or more first temperature sensors 225 may include a plurality of the first temperature sensors 225 may be included. The plurality of the first temperature sensors 225 may be disposed on the side of the membrane 220 at predetermined intervals to evenly measure the temperature of the skin of the patient 290 contacting the membrane 220. Each of the first temperature sensors 225 may include a size that does not affect and is not affected by the ultrasound waves irradiated by the transducer 210, and may be a micro-temperature sensor smaller than ¼ of an ultrasound wavelength. In other words, the size of each of the first temperature sensors 225 transparent with respect to the ultrasound waves is smaller than or equal to ¼ of the ultrasound wavelength. For example, if a frequency of an ultrasound wave is 1 MHz, and the ultrasound wave passes through a soft tissue of the body of the patient 290, a sound speed in the soft tissue may be 1540 m/s. Therefore, a wavelength of the ultrasound wave is 1.54 mm. If each of the first temperature sensors 225 includes a size smaller than 0.385 mm, that is, ¼ of 1.54 mm, each of the first temperature sensors 225 does not affect or is not affected by the ultrasound wave.

Each of the first temperature sensors 225 is included to acquire the temperature of the skin of the patient 290 contacting the membrane 220 that is used to cool the ultrasound treatment apparatus 110. Therefore, heat accumulated in the skin of the patient 290 when high power HIFU waves or consecutive ultrasound waves are irradiated is reduced, and the patient 290 receives a treatment comfortably. Also, consecutive irradiations of the consecutive ultrasound waves are enabled, and a cooling time for soothing the skin of the patient 290 is decreased, thereby reducing a total ultrasound surgical operation time.

Each of the second temperature sensors 215 measures a temperature of the transducer 210. When the transducer 210 is heated by the ultrasound waves irradiated from the transducer 210, each of the second temperature sensors 215 measures the temperature of the transducer 210. For example, high-power HIFU waves may be irradiated onto a hypervascular tumor. In this example, heat is easily generated in the transducer 210, and thus the temperature of the transducer 210 rises. The transducer 210 includes a limit temperature for an operation thereof, and ultrasound waves are controlled to be irradiated at a temperature less than the limit temperature. Therefore, the temperature of the transducer 210 is measured. The one or more second temperature sensors 215 may include a plurality of second temperature sensors 215. The plurality of the second temperature sensors 210 may be disposed on the side of the transducer 210 at predetermined intervals to evenly measure the temperature of the transducer 210.

The first data collector 232 acquires one or more temperatures T₁ of the skin of the patient 290 that are measured by the first temperature sensors 225. In other words, the first data collector 232 acquires the temperatures of the skin of the patient 290 that are measured by the first temperature sensors 225 at predetermined time intervals, and transmits the temperatures to the cooler controller 234. If the first data collector 232 acquires a plurality of the temperatures from the first temperature sensors 225, the first data collector 232 transmits, to the cooler controller 234, an average value of the acquired temperatures, a highest temperature value of the measured temperatures, and/or a temperature value measured from the first temperature sensors 225 at a position selected by a user.

The second data collector 233 acquires one or more temperatures T₂ of the transducer 210 that are measured by the second temperature sensors 215. The second data collector 233 acquires the temperatures of the transducer 210 that measured by the second temperature sensors 215 at predetermined time intervals, and transmits the temperatures to the cooler controller 234. If the second data collector 233 acquires a plurality of the temperatures of the transducer 210 from the second temperature sensors 215, the second data collector 233 transmits, to the cooler controller 234, an average value of the acquired temperatures, a highest temperature value of the measured temperatures, and/or a temperature value measured by the second temperature sensors 215 at a position selected by the user.

The cooler controller 234 controls a circulation of the cooling fluid based on the temperature of the skin of the patient 290 and/or the temperature of the transducer 210. In detail, the cooler controller 234 compares the temperature of the skin of the patient 290 or the temperature of the transducer 210 with a predetermined threshold temperature that is a cooling criterion, and determines a temperature Tᵢₙ and/or a flow rate f of the cooling fluid based on a result of the comparison to control the circulation of the cooling fluid.

For example, the cooler controller 234 compares a higher temperature value, among the temperature of the skin of the patient 290 measured by the first temperature sensors 225 and the temperature of the transducer 210 measured by the second temperature sensors 215, with the predetermined threshold temperature to determine the temperature or the flow rate of the cooling fluid. The predetermined threshold temperature may be a lower temperature among a lowest temperature at which pain is felt by heat and a limit temperature for a normal operation of the transducer 210. If the higher temperature value, among the temperature of the skin of the patient 290 measured by the first temperature sensors 225 and the temperature of the transducer 210 measured by the second temperature sensors 215, exceeds the predetermined threshold temperature, the cooler controller 234 reduces the temperature of the cooling fluid, and increases the flow rate of the cooling fluid. If the higher temperature value, among the temperature of the skin of the patient 290 measured by the first temperature sensors 225 and the temperature of the transducer 210 measured by the second temperature sensors 215, is lower than the predetermined threshold temperature, the cooler controller 234 reduces the flow rate of the cooling fluid. If the higher temperature value, among the temperature of the skin of the patient 290 measured by the first temperature sensors 225 and the temperature of the transducer 210 measured by the second temperature sensors 215, is equal to the predetermined threshold temperature, the cooler controller 234 maintains the circulation of the cooling fluid.

As another example, a plurality of predetermined threshold temperatures that are the cooling criterion may exist. For example, if a first threshold temperature is the lowest temperature inducing pain, and a second threshold temperature is the limit temperature for the normal operation of the transducer 210, the cooler controller 234 compares the temperature of the skin of the patient 290 and the temperature of the transducer 210 with the first and second threshold temperatures, respectively, to determine the temperature or the flow rate of the cooling fluid. If each of the temperature of the skin of the patient 290 and the temperature of the transducer 210 exceeds the first or second threshold temperature, the cooler controller 234 reduces the temperature of the cooling fluid, and increases the flow rate of the cooling fluid. If each of the temperature of the skin of the patient 290 and the temperature of the transducer is equal to the first and second threshold temperatures, the cooler controller 234 maintains the circulation of the cooling fluid. In other cases, the cooler controller 234 reduces the flow rate of the cooling fluid.

The cooler controller 234 acquires the temperature Tᵢₙ of the cooling fluid flowing into an inlet (in) and a temperature T_{fluid} of the cooling fluid flowing out of an outlet (out) from temperature sensors (not shown) positioned on sides of the inlet and the outlet in the space that is formed by the transducer 210 and the membrane 220 and in which the cooling fluid circulates. The cooler controller 234 checks a current flow rate of the cooling fluid from a flow rate sensor (not shown) positioned on the side of the outlet, and uses the current flow rate to determine the temperature and the flow rate of the cooling fluid.

The cooler 236 circulates the cooling fluid based on the temperature of the cooling fluid that is determined by the cooler controller 234. In other words, the cooler 236 receives the reduced temperature of the cooling fluid from the cooler controller 234, and lowers a setting temperature thereof to reduce the temperature of the cooling fluid. The cooler 236 may include a heat exchanger, a radiation fin, and a cooling fan, or may include a cooling plate to enclose a tube in which the cooling fluid flows.

The pump 238 circulates the cooling fluid based on the flow rate of the cooling fluid that is determined by the cooler controller 234. In order to circulate the cooling fluid, the pump 238 may be an infusion pump that allows the cooling fluid to flow toward the header 200 of the ultrasound treatment apparatus 110, an efflux pump that allows the cooling fluid to flow out of the header 200, or a pump into which the infusion pump and the efflux pump are integrated. If the flow rate of the cooling fluid whose temperature is reduced by the cooler 236 is increased, the cooling fluid quickly flows. Therefore, a heat exchange quickly proceeds, and thus the temperature of the transducer 210 irradiating the ultrasound waves is prevented from rising. Also, the temperature of the membrane 220 contacting the skin of the patient 290 is prevented from rising, and is further reduced to prevent the temperature of the skin of the patient 290 from rising. If the flow rate of the cooling fluid is reduced, a flow of the cooling fluid is slower than a current situation. Therefore, the heat exchange slowly proceeds, and accordingly the temperatures of the transducer 210 and the membrane 220 rise.

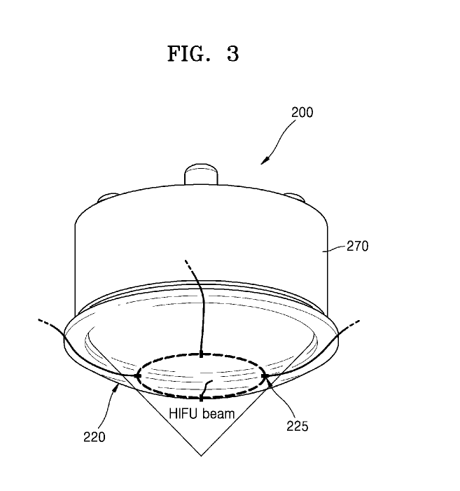

FIG. 3 is a perspective view illustrating an example of an external appearance of the header 200 of the ultrasound treatment apparatus 110. The header 200 may further include other general-purpose elements besides the elements of FIG. 3, and the external appearance of the heater 200 of the ultrasound treatment apparatus 110 may be modified.

Referring to FIG. 3, the header 200 of the ultrasound treatment apparatus 100 shown in FIG. 3 includes a housing 270, and the transducer 210 is installed in the housing 270. Since the transducer 210 includes a parabolic antenna shape, the housing 270 includes a cylindrical shape. A shape of the housing 270 may be modified based on a shape of the transducer 210. HIFU waves are irradiated from the transducer 210 of the housing 270, and HIFU beams including con-beam forms as shown in FIG. 3 are formed. The membrane 220 encloses an end of the housing 270 in a direction in which ultrasound waves are irradiated. Therefore, a space is formed by a side of the transducer 210, a side (e.g., an inside) of the membrane 220, and a part of the housing 270, and a cooling fluid circulates in the space.

Referring to FIG. 3, the first temperature sensors 225 are adhered onto another side (e.g., an outside surface) of the membrane 220 on a path of the HIFU beams including the con-beam forms, and four first temperature sensors 225 are positioned at equidistant intervals. Electric wires are connected to the first temperature sensors 225 to transmit temperature data acquired by the first temperature sensors 225.

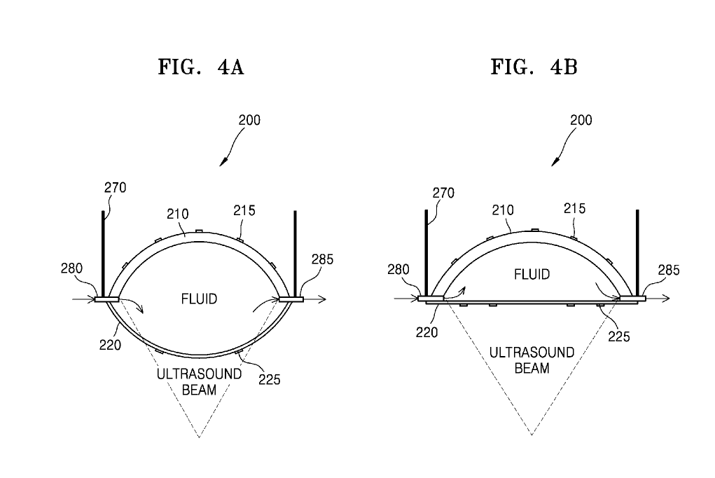

FIGS. 4A and 4B are cross-sectional views illustrating examples of the header 200 of the ultrasound treatment apparatus 110. The heater 200 may further include other general-purpose elements besides the elements of FIGS. 4A and 4B.

The cross-sectional views of the header 200 shown in FIGS. 4A and 4B are cross-sectional views of the header 200 including the housing 270 including the cylindrical shape and the transducer 210 including the parabolic antenna shape as shown in FIG. 3.

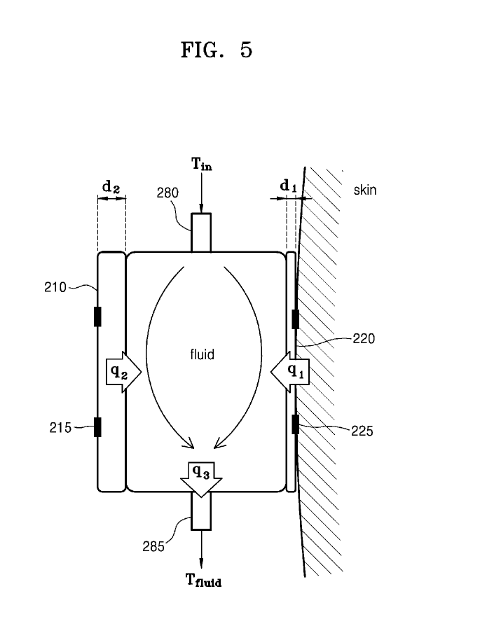

Referring to FIG. 4A, the header 200 of FIG. 4A includes the membrane 220, the first temperature sensors 225, the second temperature sensors 215, the housing 270, an inlet 280, and an outlet 285. A plurality of the second temperature sensors 215 are positioned on a side of the transducer 210, and a cooling fluid contacts another side of the transducer 210 in a direction in which ultrasound waves are irradiated. The membrane 220 is disposed in the direction in which the ultrasound waves are irradiated, to enclose an end of the housing 270. A side of the membrane 220 contacts the cooling fluid, and another side of the membrane 220 contacts the skin of the patient 290 in the direction in which the ultrasound waves are irradiated. A plurality of the first temperature sensors 225 are positioned on an area of the side of the membrane 220 that contacts the skin of the patient 290 and corresponds to an inside of an ultrasound beam formed by a path through which ultrasound waves irradiated from the transducer 210 pass. The cooling fluid flows into the inlet 280 to prevent temperatures of the transducer 210 and the membrane 220 from rising. The cooling fluid receives heat from the transducer 210 and the membrane 220 that are heated, cools the transducer 210 and the membrane 220, and is discharged through the outlet 285. A heat conduction of the cooling fluid in the header 200 is described below with reference to FIG. 5.

The membrane 220 of FIG. 4A is convex downwards A part of the membrane 220 contacting the skin of the patient 290 is dense without cracks.

Referring to FIG. 4B, the header 200 of FIG. 4B includes the transducer 210, the membrane 220, the first temperature sensors 225, the second temperature sensors 215, the inlet 280, and the outlet 285. Descriptions of the header 200 of FIG. 4B that are the same as those of the header 200 of FIG. 4A are omitted.

Differently from the membrane 220 of FIG. 4A, a part of the membrane 220 of FIG. 4B contacting the skin of the patient 290 is flat. The ultrasound treatment apparatus 110 including the header 200 as shown in FIG. 4B may be provided as a bed type. In other words, the ultrasound treatment apparatus 110 may be installed on a bed on which the patient 290 lies to irradiate ultrasound waves in an upward direction in which the patient 290 lies.

FIG. 5 is a view illustrating an example of a heat conduction generated in a header of an ultrasound treatment apparatus. A principle of a cooling effect caused by a cooling fluid is described with reference to FIG. 5. For description convenience, a heat flow flowing into and out of the cooling fluid will be described in consideration of only a heat flow q₁ [W] transmitted from the membrane 220, a heat flow q₂ transmitted from the transducer 210, and a heat flow q₃ absorbed by the cooling fluid.

The heat flow q₁ transmitted from the membrane 220 refers to a heat flow conducted to the cooling fluid through the membrane 220 based on a temperature of the skin of the patient contacting a side of the membrane 220. Referring to FIG. 5, the side of the membrane 220 contacts the skin of the patient, and the one or more first temperature sensors 225 are positioned between the membrane 220 and the skin of the patient 290 to measure the temperature of the skin. The heat flow q₁ transmitted from the membrane 220 may be expressed with the example of Equation 1 below:

$${q}_{1} = {k}_{1} \cdot {A}_{1} \cdot \frac{( {T}_{1} - {T}_{in} )}{{d}_{1}} \qquad ( 1 )$$

In Equation 1, k₁ denotes a thermal conductivity [W/m·k] of the membrane 220, A₁ denotes a contacting area [m²] of the membrane 220 contacting the cooling fluid, T₁ denotes a temperature [K] of the skin of the patient 290, Tᵢₙ denotes a temperature [K] of the cooling fluid flowing into the inlet 280, and d₁ denotes a thickness [m] of the membrane 220.

The heat flow q₂ transmitted from the transducer 210 refers to a heat flow conducted from the transducer 210 to the cooling fluid. Referring to FIG. 5, the one or more second temperature sensors 215 are positioned on a side of the transducer 210 that is heated, to measure a temperature of the transducer 210, and the cooling fluid contacts another side of the transducer 210. The heat flow q₂ transmitted from the transducer 210 may be expressed with the example of Equation 2 below:

$${q}_{2} = {k}_{2} \cdot {A}_{2} \cdot \frac{( {T}_{2} - {T}_{in} )}{{d}_{2}} \qquad ( 2 )$$

In Equation 2, k₂ denotes a thermal conductivity of the transducer 210, A denotes a contact area of the transducer 210 contacting the cooling fluid, T₂ denotes a temperature of the transducer 210, Tᵢₙ denotes the temperature of the cooling fluid that flows in, and d₂ denotes a thickness of the transducer 210.

The heat flow q₃ absorbed by the cooling fluid refers to a heat flow transmitted from the membrane 220 and the transducer 210, and may be expressed with the example of Equation 3 below:

q₃=f·c·(T_{fluid}−Tᵢₙ) (3)

In Equation 3, f denotes a flow rate [L/s] of the cooling fluid, c denotes a specific heat [Kcal/Kg·K], T_{fluid} denotes a temperature of the cooling fluid discharged through the outlet 285, and Tᵢₙ denotes the temperature of the cooling fluid that flows in.

The example of Equation 4 below is established between the heat flow q₁ transmitted from the membrane 220, the heat flow q₂ transmitted from the transducer 210, and the heat flow q₃ absorbed by the cooling fluid based on the principle of the conservation of energy:

q₃=q₁+q₂ (4)

If both the temperature T₁ of the skin of the patient 290 contacting the membrane 220 and the temperature T₂ of the transducer 210 increase, a value of a right hand side of Equation 4 above increases. Therefore, a left hand value of Equation 4 increases to establish Equation 4 above. Referring to Equation 3, the flow rate f of the cooling fluid and the temperature Tᵢₙ of the cooling fluid that flows in are preset values, and the specific heat c of the cooling fluid is a characteristic value that does not vary. Therefore, the temperature T_{fluid} of the cooling fluid discharged through the outlet 285 increases. However, setting of the flow rate f of the cooling fluid that is the preset value and the temperature Tᵢₙ of the cooling fluid that flows in may be changed to achieve a cooling effect due to the cooling fluid. The cooling unit 230 adjusts the temperature Tᵢₙ and the flow rate f of the cooling fluid to control the temperature T₂ of the transducer 210 and the temperature T₁ of the skin of the patient 290 based on the cooling effect achieved by the cooling fluid. To do so, the temperature T₁ of the skin of the patient 290 contacting the membrane 220 and the temperature T₂ of the transducer 210 are respectively measured by the first and second temperature sensors 225 and 215, respectively.

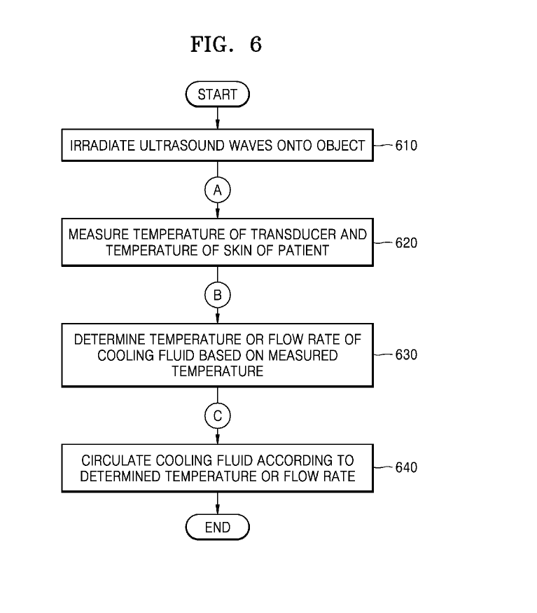

FIG. 6 is a flowchart illustrating an example of a method of cooling an ultrasound treatment apparatus. Though omitted below, the descriptions of the ultrasound treatment apparatus 100 also apply to the method of FIG. 6.

In operation 610, ultrasound waves are irradiated onto an object. In more detail, the ultrasound waves generated from the transducer 210 are irradiated onto the object including a lesion. The ultrasound waves may be HIFU waves.

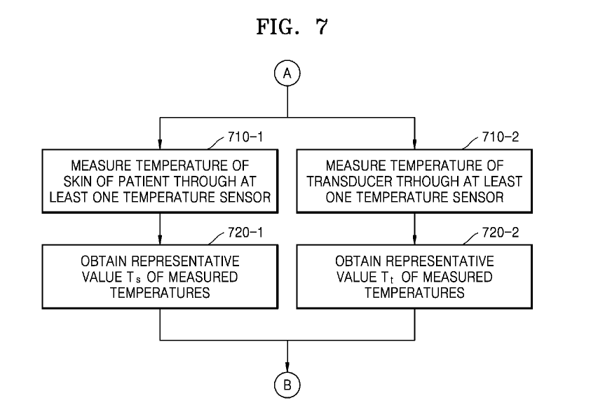

In operation 620, a temperature of the transducer 210 and a temperature of a skin of the patient 290 are measured. The temperature of the transducer 210 and the temperature of the skin of the patient 290 contacting the membrane 220 may be simultaneously measured using the first and second temperature sensors 215 and 225, respectively. To measure the temperature of the skin of the patient 290 contacting the membrane 220, the first temperature sensors 215 may directly contact the skin of the patient 290 on an irradiation path of the ultrasound waves. The operation of measuring the temperature of the skin of the patient 290 contacting the membrane 220 and the temperature of the transducer 210 is described with reference to FIG. 7.

FIG. 7 is a detailed flowchart illustrating an example of operation 620 of FIG. 6. Assuming that the first and second temperature sensors 215 and 225 measure the temperature of the skin of the patient 290 contacting the membrane 220 and the temperature of the transducer 210, a measurement of the temperatures will now be described.

In operation 710-1, the temperature of the skin of the patient 290 is measured through at least one of the first temperature sensors 225. If a plurality of the first temperature sensors 225 is included, the first temperature sensors 225 may be positioned at predetermined intervals on the membrane 220.

In operation 720-1, the first data collector 232 obtains a representative value Tₛ of the measured temperature of the skin of the patient 290. If one of the first temperature sensors 225 is included, the measured temperature is the representative value Tₛ. If a plurality of the first temperature sensors 225 is included, the representative value Ts may be an average value of measured temperatures of the skin of the patient 290, the highest temperature value of the measured temperatures, or a temperature value measured by the first temperature sensors 225 at a position selected by an user.

In operation 710-2, a temperature of the transducer 210 is measured by through at least one of the second temperature sensors 215. If a plurality of the second temperature sensors 215 is included, the second temperature sensors 215 may be positioned at predetermined intervals on the transducer 210.

In operation 720-2, the second data collector 233 obtains a representative value Tₜ of the measured temperature of the transducer 210. If one of the second temperature sensors 215 is included, the measured temperature value is the representative value Tₜ. If a plurality of the second temperature sensors 215 is included, the representative value Tₜ may be an average value of measured temperatures of the transducer 210, the highest temperature value of the measured temperatures, or a temperature value measured by the second temperature sensors 215 at a position selected by the user.

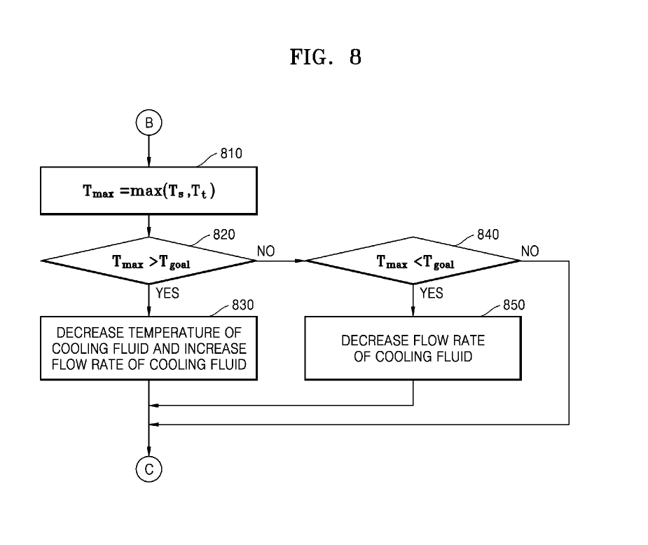

Referring to FIG. 6 again, in operation 630, a temperature or a flow rate of the cooling fluid is determined based on the temperature of the transducer 210 and/or the temperature of the skin of the patient 290. In other words, the temperature of the transducer 210 and/or the temperature of the skin of the patient 29 is compared with a predetermined threshold temperature that is a cooling criterion, and the temperature and/or the flow rate of the cooling fluid is determined based on a result of the comparison. The predetermined threshold temperature that is the cooling criterion may be a lower temperature among a limit temperature for a normal operation of the transducer 210 and a lowest temperature causing pain because of heat. The operation of determining the temperature or the flow rate of the cooling fluid will now be described with reference to FIG. 8.

FIG. 8 is a detailed flowchart illustrating an example of operation 630 of FIG. 6. The operation of determining the temperature or the flow rate of the cooling fluid is illustrated in FIG. 8, and is not limited thereto.

In operation 810, a higher (i.e., maximum) temperature among the temperature Tₛ of the skin of the patient 290 and the temperature Tₜ of the transducer 210 is selected as a temperature Tₘₐₓ to be compared with the predetermined threshold temperature that is the cooling criterion. If a plurality of the first temperature sensors 225 and a plurality of the second temperature sensors 215 are included, the representative value Tₛ of the temperature of the skin of the patient 290 that is obtained by the first data collector 232 is compared with the representative value Tₜ of the temperature of the transducer 210 that obtained by the second data collector 233 to select the higher temperature among the temperatures Tₛ and Tₜ.

In operation 820, it is determined whether the selected temperature Tₘₐₓ is greater than the predetermined threshold temperature T_{goal}. The predetermined threshold temperature T_{goal} may be a lower temperature among a lowest temperature causing pain due to feeling heat and a limit temperature for a normal operation of the transducer 210. If the selected temperature Tₘₐₓ is determined to be greater than the predetermined threshold temperature T_{goal}, the method continues in operation 830. Otherwise, the method continues in operation 840.

In operation 830, the temperature of the cooling fluid is decreased, and the flow rate of the cooling fluid is increased.

In operation 840, it is determined whether the selected temperature Tₘₐₓ is less than the predetermined threshold temperature T_{goal}. If the selected temperature Tₘₐₓ is determined to be less than the predetermined threshold temperature T_{goal}, the method continues in operation 850. Otherwise, the method returns to FIG. 6. That is, the selected temperature value Tₘₐₓ is equal to the predetermined threshold temperature T_{goal}, and a current circulation of the cooling fluid is maintained

In operation 850, the flow rate of the cooling fluid is decreased

Differently from shown in FIG. 8, if a plurality of predetermined threshold temperatures that are the cooling criterion exist, e.g., a first threshold temperature is a lowest temperature causing pain due to feeling heat, and a second threshold temperature is a limit temperature for a normal operation of the transducer 210, operation 810 of FIG. 8 is not necessary. Also, in operations 820 and 840 of FIG. 8, the representative value Tₛ of the temperature of the skin of the patient 290 obtained by the first data collector 232 and the representative value Tₜ of the temperature of the transducer 210 obtained by the second data collector 233 are respectively compared with the first and second threshold temperatures to determine a temperature or a flow rate of the cooling fluid. If each of the temperatures Tₛ and Tₜ exceeds the first or second threshold temperature, the temperature of the cooling fluid is decreased and the flow rate of the cooling fluid is increased. If each of the temperatures Tₛ and Tₜ is equal to the first or second threshold temperature, the current circulation of the cooling fluid is maintained. In other cases, the flow rate of the cooling fluid is decreased.

Referring to FIG. 6 again, in operation 640, the cooling fluid is circulated according to the determined temperature or the flow rate of the cooling fluid. A setting temperature of the cooler 236 may be lowered to decrease the temperature of the cooling fluid. A power of the pump 238 may be increased and decreased to circulate the cooling fluid in order to increase and decrease, respectively, the flow rate of the cooling fluid.

The examples of an ultrasound treatment apparatus described may maintain uniform a temperature of a transducer and a temperature of the skin of a patient contacting the ultrasound treatment apparatus.

The various units, elements, and methods described above may be implemented using one or more hardware components, one or more software components, or a combination of one or more hardware components and one or more software components.

A hardware component may be, for example, a physical device that physically performs one or more operations, but is not limited thereto. Examples of hardware components include microphones, amplifiers, low-pass filters, high-pass filters, band-pass filters, analog-to-digital converters, digital-to-analog converters, and processing devices.

A software component may be implemented, for example, by a processing device controlled by software or instructions to perform one or more operations, but is not limited thereto. A computer, controller, or other control device may cause the processing device to run the software or execute the instructions. One software component may be implemented by one processing device, or two or more software components may be implemented by one processing device, or one software component may be implemented by two or more processing devices, or two or more software components may be implemented by two or more processing devices.

A processing device may be implemented using one or more general-purpose or special-purpose computers, such as, for example, a processor, a controller and an arithmetic logic unit, a digital signal processor, a microcomputer, a field-programmable array, a programmable logic unit, a microprocessor, or any other device capable of running software or executing instructions. The processing device may run an operating system (OS), and may run one or more software applications that operate under the OS. The processing device may access, store, manipulate, process, and create data when running the software or executing the instructions. For simplicity, the singular term “processing device” may be used in the description, but one of ordinary skill in the art will appreciate that a processing device may include multiple processing elements and multiple types of processing elements. For example, a processing device may include one or more processors, or one or more processors and one or more controllers. In addition, different processing configurations are possible, such as parallel processors or multi-core processors.

A processing device configured to implement a software component to perform an operation A may include a processor programmed to run software or execute instructions to control the processor to perform operation A. In addition, a processing device configured to implement a software component to perform an operation A, an operation B, and an operation C may include various configurations, such as, for example, a processor configured to implement a software component to perform operations A, B, and C; a first processor configured to implement a software component to perform operation A, and a second processor configured to implement a software component to perform operations B and C; a first processor configured to implement a software component to perform operations A and B, and a second processor configured to implement a software component to perform operation C; a first processor configured to implement a software component to perform operation A, a second processor configured to implement a software component to perform operation B, and a third processor configured to implement a software component to perform operation C; a first processor configured to implement a software component to perform operations A, B, and C, and a second processor configured to implement a software component to perform operations A, B, and C, or any other configuration of one or more processors each implementing one or more of operations A, B, and C. Although these examples refer to three operations A, B, C, the number of operations that may implemented is not limited to three, but may be any number of operations required to achieve a desired result or perform a desired task.

Software or instructions that control a processing device to implement a software component may include a computer program, a piece of code, an instruction, or some combination thereof, that independently or collectively instructs or configures the processing device to perform one or more desired operations. The software or instructions may include machine code that may be directly executed by the processing device, such as machine code produced by a compiler, and/or higher-level code that may be executed by the processing device using an interpreter. The software or instructions and any associated data, data files, and data structures may be embodied permanently or temporarily in any type of machine, component, physical or virtual equipment, computer storage medium or device, or a propagated signal wave capable of providing instructions or data to or being interpreted by the processing device. The software or instructions and any associated data, data files, and data structures also may be distributed over network-coupled computer systems so that the software or instructions and any associated data, data files, and data structures are stored and executed in a distributed fashion.

For example, the software or instructions and any associated data, data files, and data structures may be recorded, stored, or fixed in one or more non-transitory computer-readable storage media. A non-transitory computer-readable storage medium may be any data storage device that is capable of storing the software or instructions and any associated data, data files, and data structures so that they can be read by a computer system or processing device. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, or any other non-transitory computer-readable storage medium known to one of ordinary skill in the art.

Functional programs, codes, and code segments that implement the examples disclosed herein can be easily constructed by a programmer skilled in the art to which the examples pertain based on the drawings and their corresponding descriptions as provided herein.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An ultrasound treatment apparatus comprising:
a transducer configured to irradiate ultrasound waves onto an object;
a membrane disposed in an irradiation direction of the ultrasound waves and to form, with the transducer, a space in which a cooling fluid circulates;
a first temperature sensor configured to measure a temperature of a skin of a patient contacting the membrane;
a second temperature sensor configured to measure a temperature of the transducer; and
a cooling unit configured to circulate the cooling fluid based on the measured temperatures.

2. The ultrasound treatment apparatus of claim 1, wherein the first temperature sensor is disposed on a side of the membrane directly contacting the skin, and inside an ultrasound beam formed by an irradiation path of the ultrasound waves.

3. The ultrasound treatment apparatus of claim 1, wherein:
the first temperature sensor comprises first temperature sensors disposed at predetermined intervals on the membrane; and
the second temperature sensor comprises second temperature sensors disposed at predetermined intervals on the transducer.

4. The ultrasound treatment apparatus of claim 1, wherein a size of the first temperature sensor is less than or equal to ¼ of a size of a wavelength of the ultrasound waves.

5. The ultrasound treatment apparatus of claim 1, wherein the cooling unit comprises:
a cooler controller configured to compare the measured temperature of the transducer or the measured temperature of the skin with a predetermined temperature that is a cooling criterion, and determine a temperature and/or a flow rate of the cooling fluid based on a result of the comparison; and
a cooler configured to circulate the cooling fluid based on the determined temperature and/or the determined flow rate.

6. The ultrasound treatment apparatus of claim 5, wherein the cooler controller is configured to:
compare a higher temperature among the temperature of the skin and the temperature of the transducer with the predetermined temperature;
decrease the temperature of the cooling fluid, and increase the flow rate, in response to the higher temperature being greater than the predetermined temperature;
decrease the flow rate in response to the higher temperature being less than the predetermined temperature; and
maintain a current circulation of the cooling fluid in response to the higher temperature being equal to the predetermined temperature.

7. The ultrasound treatment apparatus of claim 6, wherein the predetermined temperature is a lower temperature among a lowest temperature causing pain due to feeling heat and a limit temperature for a normal operation of the transducer.

8. The ultrasound treatment apparatus of claim 5, wherein the cooler controller is configured to:
compare the temperature of the skin and the temperature of the transducer with first and second predetermined temperatures, respectively;
decrease the temperature of the cooling fluid, and increase the flow rate, in response to each of the temperature of the skin and the temperature of the transducer being greater than the first or second predetermined temperature;
maintain a current circulation of the cooling fluid in response to each of the temperature of the skin and the temperature of the transducer being equal to the first or second predetermined temperature; and
decrease the flow rate in other cases.

9. The ultrasound treatment apparatus of claim 8, wherein:
the first predetermined temperature is a lowest temperature causing pain due to feeling heat; and
the second predetermined temperature is a limit temperature for a normal operation of the transducer.

10. The ultrasound treatment apparatus of claim 1, wherein the cooling unit comprises:
a first data collector configured to acquire one or more temperatures of the skin that are measured by the first temperature sensor, and obtain, as a representative value of the temperature of the skin, an average of the acquired one or more temperatures of the skin, or a highest temperature among the acquired one or more temperatures of the skin, or a temperature value of the skin that is measured by a temperature sensor at a position selected by a user; and
a second data collector configured to acquire one or more temperatures of the transducer that are measured by the second temperature sensor, and obtain, as a representative value of the temperature of the transducer, an average of the acquired one or more temperatures of the transducer, or a highest temperature among the acquired one or more temperatures of the transducer, or a temperature value of the transducer that is measured by another temperature sensor at another position selected by the user.

11. A method of cooling an ultrasound treatment apparatus comprising a transducer irradiating ultrasound waves onto an object, and a membrane forming, with the transducer, a space in which a cooling fluid circulates, the method comprising:
measuring a temperature of a skin of a patient contacting the membrane;
measuring a temperature of the transducer; and
circulating the cooling fluid based on the measured temperatures.

12. The method of claim 11, wherein the measuring of the temperature of the skin comprises measuring the temperature of the skin at a side of the membrane directly contacting the skin, and inside an ultrasound beam formed by an irradiation path of the ultrasound waves.

13. The method of claim 11, wherein:
the measuring of the temperature of the skin comprises measuring temperatures of the skin;
the measuring of the temperature of the transducer comprises measuring temperatures of the transducer; and
the method further comprises
obtaining, as a representative value of the temperature of the skin, an average of the measured temperatures of the skin, or a highest temperature among the measured temperatures of the skin, or a temperature value of the skin that is measured by a temperature sensor at a position selected by a user, and
obtaining, as a representative value of the temperature of the transducer, an average of the measured temperatures of the transducer, or a highest temperature among the measured temperatures of the transducer, or a temperature value of the transducer that is measured by another temperature sensor at another position selected by the user.

14. The method of claim 11, wherein the circulating comprises:
comparing the temperature of the transducer or the temperature of the skin with a predetermined temperature that is a cooling criterion;
determining a temperature and/or a flow rate of the cooling fluid based on a result of the comparing; and
circulating the cooling fluid based on the determined temperature and/or the determined flow rate.

15. The method of claim 14, wherein:
the comparing comprises comparing a higher temperature among the temperature of the skin and the temperature of the transducer with the predetermined temperature; and
the determining comprises
decreasing the temperature of the cooling fluid, and increasing the flow rate, in response to the higher temperature being greater than the predetermined temperature;
decreasing the flow rate in response to the higher temperature being less than the predetermined temperature; and
maintaining a current circulation of the cooling fluid in response to the higher temperature being equal to the predetermined temperature.

16. The method of claim 15, wherein the predetermined temperature is a lower temperature among a lowest temperature causing pain due to feeling heat and a limit temperature for a normal operation of the transducer.

17. The method of claim 14, wherein:
the comparing comprises comparing the temperature of the skin and the temperature of the transducer with first and second predetermined temperatures, respectively; and
the determining comprises
decreasing the temperature of the cooling fluid, and increasing the flow rate, in response to each of the temperature of the skin and the temperature of the transducer being greater than the first or second predetermined temperature,
maintain a current circulation of the cooling fluid in response to each of the temperature of the skin and the temperature of the transducer being equal to the first or second predetermined temperature, and
decreasing the flow rate in other cases.

18. The method of claim 17, wherein:
the first predetermined temperature is a lowest temperature causing pain due to feeling heat; and
the second predetermined temperature is a limit temperature for a normal operation of the transducer.

19. A non-transitory computer-readable storage medium storing a program comprising instructions to cause a computer to perform the method of claim 11.

20. An apparatus comprising:
a transducer configured to irradiate an ultrasound beam onto a subject; and
a membrane configured to form, with the transducer, a space in which a cooling fluid circulates, and contact the subject.
